(19) United States
(12) Patent Application Publication
(10) Pub. No.: US 2026/0308033 A1
(43) Pub. Date: Oct. 8, 2026

(54) Microbial Composite Bacteria for Sludge Reduction in Biochemical Systems, Preparation Method Thereof, and Application of Microbial Agent for Sludge Reduction in Biochemical Systems

(71) Applicant: MicIntel Dynamics Inc., Waterloo (CA)

(72) Inventors: Jie Wen, Beijing (CN); Xiaoxi Shu, Beijing (CN)

(21) Appl. No.: 19/641,142

(22) Filed: Apr. 7, 2026

(30) Foreign Application Priority Data
Apr. 7, 2025 (CN) 202510428151.5

(51) Int. Cl. C02F 3/34; C02F 3/12; C02F 11/02; C12N 1/20

(52) U.S. Cl. CPC C02F 3/341; C02F 3/1263; C02F 11/02; C12N 1/20

(57) ABSTRACT

The present invention discloses a microbial composite bacterium for sludge reduction in biochemical systems, a preparation method thereof, and an application of a microbial inoculum for sludge reduction in biochemical systems. The invention belongs to the field of microorganisms and addresses the problem of how to achieve reduction of excess sludge generated during wastewater treatment at low cost without causing secondary pollution or producing solid waste, The present invention discloses a microbial composite and agent composed of Bacillus smithii (Preservation No.: CGMCC No. 31841), Heyndrickxia coagulans (Preservation No.: CGMCC No. 31842), Parageobacillus caldoxylosilyticus (Preservation No.: CGMCC No. 31844), Aeribacillus pallidus (Preservation No.: CGMCC No. 31845), and Pallidibacillus thermolactis (Preservation No.: CGMCC No. 31847). These are applied in biochemical systems to achieve reduction of excess sludge in wastewater treatment at the source. The invention offers advantages such as high activity, low investment, reduced energy consumption, convenient operation, and no secondary pollution.

### TECHNICAL FIELD

The present invention relates to the field of microbial technology, and in particular to a composite microbial Bacteria for sludge reduction in biochemical systems, its preparation method, and the application of the microbial agent for sludge reduction in biochemical systems.

### INTRODUCTION

Sludge is generally a byproduct of sewage treatment, and it is an extremely complex heterogeneous substance composed of organic debris, bacterial cells, inorganic particles, colloids, and other components. The main characteristics of sludge are high water content (which can be as high as over 99%), high organic matter content, proneness to decomposition and odor generation, as well as fine particle size, low specific gravity, and a colloidal liquid state.

In environmental engineering, the sludge to be treated generally refers to the precipitated substances generated during the sewage treatment process in sewage treatment plants and the scum floating on the surface of sewage. It has a complex composition, containing a large number of microorganisms, pathogens, heavy metals, and organic pollutants. Due to the large amount of harmful substances it contains, improper disposal will result in secondary pollution from the sewage treatment plant.

As pointed out in the 14th Five-Year Plan for the Development of Urban Sewage Treatment and Resource Utilization jointly issued by the National Development and Reform Commission (NDRC) and the Ministry of Housing and Urban-Rural Development (MOHURD), the national target for the sewage treatment rate in counties is to reach over 95% by 2025. In contrast, the safe disposal rate of sludge—its byproduct—remains relatively low, and the phenomenon of prioritizing water treatment while neglecting sludge management is quite prominent. In recent years, sludge disposal has gradually gained attention, and the state has issued a series of policies and measures. For example, the Action Plan for Water Pollution Prevention and Control (commonly known as the “Water Ten Measures”) issued by the State Council in 2015 clearly stipulates: “Existing sludge treatment and disposal facilities shall basically complete compliance renovation by the end of 2017, and the harmless treatment and disposal rate of sludge in prefecture-level and above cities shall reach over 90% by the end of 2020.”.

Since the sludge disposal industry in China started relatively late, the independently developed sludge treatment and disposal technologies are still being continuously improved. Therefore, the sludge disposal rate remains extremely low-less than 35% of the national sludge was treated in 2016.

At present, the current status of sludge treatment in China is as follows:
I. Sanitary Landfill: As the most commonly used traditional sludge disposal method, landfilling is simple, feasible, and low-cost. However, it fails to achieve the ultimate disposal goals of sludge, such as harmlessness, stabilization, reduction, and resource utilization, and is prone to causing secondary pollution of sludge. In addition, due to the current shortage of land resources, landfills are gradually being replaced by incineration plants, and this disposal method will also face elimination.
II. Land Application: The main forms of sludge land application include agricultural use of sludge, application of sludge for the improvement of forests, horticultural areas, abandoned mines, and other sites. However, since sludge also contains toxic and harmful microorganisms and heavy metals, it must undergo stabilization and harmless treatment prior to land application; otherwise, it will cause secondary pollution to the soil.
III. Sludge Incineration: This method refers to the combustion of sludge mixed with certain auxiliary fuels in a specific proportion. Sludge incineration can achieve the goal of reduction, with organic matter completely oxidized and heavy metals almost remaining in the ash residue. However, the waste gas generated from sludge incineration will cause secondary pollution to the environment. Secondly, due to the high water content of sludge, its combustion calorific value is low, requiring the addition of a large amount of auxiliary fuels, which is neither economical nor reasonable.
IV. Sludge Composting: Composting is a process that utilizes microorganisms in sludge for fermentation. A certain proportion of bulking agents and conditioners such as straw, rice straw, wood chips, or domestic waste are added to the sludge, and microbial communities oxidize and decompose various organic substances in a humid environment, converting them into humus-like substances. Humus-like substances are a type of high-quality organic compound fertilizer or can be used to produce organic bacterial fertilizer.
V. Sludge Thermal Drying: Thermal drying refers to the process of drying sludge using thermal energy. Its high-temperature sterilization effect can kill pathogens and parasite eggs, enabling rapid sludge drying and avoiding the impact of odors on the surrounding environment. The volume reduction of sludge after thermal drying is particularly significant, with the volume being reducible by 75-80%. The resulting high-efficiency granular fertilizer is easy to store and transport.

Of course, there are also new technologies for sludge treatment in China at present. For example, the low-temperature solar composite membrane sludge treatment technology uniformly distributes sludge with a water content of 80%-85% into a closed solar composite hydrophobic membrane box, using solar energy as the energy source for low-temperature evaporation and deep drying to obtain sludge dry residue with a water content of <8%. The dry residue can be used to prepare activated carbon, and the evaporated water can be reused or discharged up to standard. Biomass co-firing power generation refers to the co-firing power generation of biomass resources such as sludge in existing coal-fired power plants, which can promote the reduction, harmlessness, resource utilization and large-scale disposal of sludge. The above methods all focus on how to treat sludge or reduce it through physical and chemical means, and fail to achieve sludge reduction at the source.

### SUMMARY

To solve the technical problem of reducing excess sludge from the source during sewage treatment at low cost without generating secondary pollution or solid waste by preparing a composite microbial Bacteria or microbial agent for sludge reduction in biochemical systems, the present invention provides a composite microbial Bacteria for sludge reduction in biochemical systems, its preparation method, and the application of the microbial agent for sludge reduction in biochemical systems. The present invention applies microbial fermentation technology to biochemical systems, realizing the reduction of excess sludge during sewage treatment from the source. This technology improves the efficiency of sludge reduction, reduces operating costs, solves the problems of large investment and environmental pollution, and truly achieves a technological revolution in low-cost, harmless and environmentally friendly sludge treatment. It has extremely broad application prospects in sewage treatment and sludge treatment.

The objective of the present invention is specifically achieved through the following technical solutions:

The present invention discloses Bacillus smithii, which is characterized by the deposit number of CGMCC No. 31841, the deposit date of Sep. 3, 2024, the depositary institution being China General Microbiological Culture Collection Center (CGMCC), and the address located at No. 3, Courtyard 1, Beichen West Road, Chaoyang District, Beijing, China.

The present invention discloses Heyndrickxia coagulans, which is characterized by a deposit number of CGMCC No. 31842, a deposit date of Sep. 3, 2024, a depositary institution of China General Microbiological Culture Collection Center (CGMCC), and an address of No. 3, Courtyard 1, Beichen West Road, Chaoyang District, Beijing, China.

The present invention discloses Parageobacillus caldoxylosilyticus, which is characterized by a deposit number of CGMCC No. 31844, a deposit date of Sep. 3, 2024, a depositary institution of China General Microbiological Culture Collection Center (CGMCC), and an address of No. 3, Courtyard 1, Beichen West Road, Chaoyang District, Beijing, China.

The present invention discloses Aeribacillus pallidus, which is characterized by a deposit number of CGMCC No. 31845, a deposit date of Sep. 3, 2024, a depositary institution of China General Microbiological Culture Collection Center (CGMCC), and an address of No. 3, Courtyard 1, Beichen West Road, Chaoyang District, Beijing, China.

The present invention discloses Pallidibacillus thermolactis, which is characterized by a deposit number of CGMCC No. 31847, a deposit date of Sep. 3, 2024, a depositary institution of China General Microbiological Culture Collection Center (CGMCC), and an address of No. 3, Courtyard 1, Beichen West Road, Chaoyang District, Beijing, China.

The present invention discloses a composite microbial Bacteria for sludge reduction in biochemical systems, comprising Bacillus smithii, Heyndrickxia coagulans, Parageobacillus caldoxylosilyticus, Aeribacillus pallidus, and Pallidibacillus thermolactis.

Among them, the ratio of effective viable counts of Bacillus smithii:Heyndrickxia coagulans:Parageobacillus caldoxylosilyticus:Aeribacillus pallidus:Pallidibacillus thermolactis is (10%-25%):(25%-30%):(18%-26%):(12%-29%):(9%-30%).

The invention discloses a preparation method of a microbial composite bacteria for sludge reduction in biochemical systems, comprising:
Step 1: Add sterile water to a sterile container containing soil material, thoroughly mix the soil material with the sterile water, and then allow the mixture to stand for sedimentation; wherein the soil material is surface moist soil or wet silt with apparent humus from a riverbank that has accumulated for over 20 years;
Step 2: Take the supernatant, perform serial dilution on the supernatant using the dilution plating method, then separately take the suspension of the supernatant at each dilution gradient and evenly spread it on NB solid medium, LB solid medium, NA solid medium, TSA solid medium, and PDA solid medium. Place these media in an incubator for constant-temperature cultivation at 40° C. Additionally, take the undiluted supernatant, evenly spread it on NB solid medium, LB solid medium, NA solid medium, TSA solid medium, and PDA solid medium (serving as the control group), and place it in an incubator for constant-temperature cultivation at 37° C.;
Step 3: After the formation of single colonies, observe the bacterial separation across all dilution gradient plates. Visually identify and select one target gradient where the separation effectiveness meets the requirements. Select colonies with different morphological appearances from the target gradient, pick the single colonies, perform serial dilution on the single colonies using the dilution plating method, then evenly spread them on the corresponding medium, and place the medium in an incubator for constant-temperature cultivation at 35° C. For the control group, evenly spread the undiluted single colonies on the corresponding medium, place them in the incubator for constant-temperature cultivation at 35° C., and then increase the incubator temperature by 1° C. incrementally. Repeat this step until the incubator temperature reaches 45° C. Repeat this entire process again until the morphological characteristics of each single colony become consistent;
Step 4: Use the streaking method for isolation to purify the endophytic bacteria and obtain monoclonal colonies. After 16S rDNA gene sequencing, the microbial composite bacterium for sludge reduction in biochemical systems is obtained.

In Step 1, the preferred soil materials are: riparian wetland soil from humic alluvial soil or organic gley soil.

In Step 3, the formula of the corresponding culture medium includes:

Formulation of the Culture Medium for Bacillus smithii:
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;
   Formulation of the Culture Medium for Heyndrickxia coagulans:
NB Solid Medium: Peptone 20 g/L, Beef Extract 3 g/L, Agar Powder 20 g/L;
NB Liquid Medium: Peptone 20 g/L, Beef Extract 3 g/L;
After preparation, sterilize by autoclaving at 121° C. for 20 minutes;
   Formulation of the Culture Medium for Parageobacillus caldoxylosilyticus:
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;
   Formulation of the Culture Medium for Aeribacillus pallidus:
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;
   Formulation of the Culture Medium for Pallidibacillus thermolactis:
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;

The invention discloses a microbial agent for sludge reduction in biochemical systems, comprising Bacillus smithii, Heyndrickxia coagulans, Parageobacillus caldoxylosilyticus, Aeribacillus pallidus, Pallidibacillus thermolactis, and a microbial composite bacteria for sludge reduction in biochemical systems (or the microbial composite bacteria for sludge reduction in biochemical systems obtained by a preparation method).

The invention discloses the application of Bacillus smithii, Heyndrickxia coagulans, Parageobacillus caldoxylosilyticus, Aeribacillus pallidus, Pallidibacillus thermolactis, a microbial composite bacteria for sludge reduction in biochemical systems, the microbial composite bacteria for sludge reduction in biochemical systems obtained by a preparation method, or a microbial agent for sludge reduction in biochemical systems in the sludge reduction of biochemical systems.

The present invention discloses a microbial composite bacterium for biochemical system sludge reduction, a method for preparing the same, the microbial composite bacterium for biochemical system sludge reduction obtained by the preparation method, or a method for using a microbial agent for biochemical system sludge reduction in a biochemical system, comprising:

Take the composite microbial bacteria for sludge reduction in biochemical systems or the microbial agent for sludge reduction in biochemical systems and add it to the biochemical system to mix with the sludge.

Among them, the dosing methods include:
The temperature of the biochemical system is maintained at 25° C. to 45° C., the DO (Dissolved Oxygen) value is between 1.0 mg/L and 4.0 mg/L, and the pH value ranges from 6 to 9;
The dosing points are: the inlet end of the aerobic tank in the biochemical system and the sludge return point of the secondary clarifier;
To reduce 1.0 ton of absolute dry sludge in the biochemical system, the dosage of the composite microbial bacteria for sludge reduction in biochemical systems or the microbial agent for sludge reduction in biochemical systems is 2.5~5 kg;
Determine the daily dosage of the microbial composite bacteria for sludge reduction in biochemical systems or the microbial agent for sludge reduction in biochemical systems based on the excess sludge production derived from the daily influent load and output of the biochemical system.

Among them, the temperature at which the microbial composite bacteria for sludge reduction in biochemical systems or the microbial agent for sludge reduction in biochemical systems exhibit the strongest microbial activity ranges from 35° C. to 45° C., and the preferred temperature of the biochemical system is 35° C. to 45° C.

The beneficial effects of the present invention are as follows:

Through extensive experimental verification, the ability of the microbial strains in the microbial composite bacteria for sludge reduction in biochemical systems or the microbial agent for sludge reduction in biochemical systems disclosed in the present invention to secrete different enzymes under specific environments is as follows:
Bacillus smithii secretes protease and amylase;
Heyndrickxia coagulans secretes L-lactate dehydrogenase, amylase, and protease;
Parageobacillus caldoxylosilyticus secretes xylanase and cellulase;
Aeribacillus pallidus secretes 4-α-glucosyltransferase, amylase, and protease;
Pallidibacillus thermolactis secretes glycolytic enzymes and lactate dehydrogenase.

The aforementioned microbial strains and different enzymes act to decompose various component substances of aged or dead microbial cells in the biochemical system, thereby providing energy for the microbial composite bacteria/microbial agent. The decomposed organic matrix of the sludge microbial cells produces carbon dioxide and water, or partially adsorbed organic matrices in the sludge are decomposed and converted into carbon dioxide and water, thus achieving sludge reduction. Meanwhile, through microbial competition, the growth of certain undesirable bacteria, such as filament-producing microorganisms, is inhibited.

By adding the microbial composite bacteria for sludge reduction in biochemical systems or the microbial agent for sludge reduction in biochemical systems disclosed in the present invention into the biochemical system, the present invention reduces the produced excess sludge, thereby achieving sludge reduction control at the source. The technical solution disclosed in the present invention has the following advantages:
1. No hardware investment is required, featuring extremely low energy consumption and relatively low investment and operation costs;
2. It can reduce the excess sludge in the biochemical system by 30% to 80%;
3. For systems requiring additional carbon sources, the supplementary amount of carbon sources can be reduced;
4. The sludge after reduction is more amenable to dewatering or disposal.

The technical solution disclosed in the present invention is applicable to sewage treatment plants handling substances with high organic content and no obvious toxicity. It is also suitable for the centralized disposal of multiple sewage plants and the co-treatment of various organic wastes, featuring high activity, low investment, low energy consumption, convenient operation, and no secondary pollution.

### Biological Deposit Statement:

(1) Biological Material: SW-B101, Taxonomic Name: Bacillus smithii, Deposit Number: CGMCC No. 31841, Deposit Date: Sep. 3, 2024, Depository Authority: China General Microbiological Culture Collection Center (CGMCC), Address: Institute of Microbiology, Chinese Academy of Sciences, No. 3, Courtyard 1, Beichen West Road, Chaoyang District, Beijing, China.
(2) Biological Material: SW-B102, Taxonomic Name: Heyndrickxia coagulans, Deposit Number: CGMCC No. 31842, Deposit Date: Sep. 3, 2024, Depository Authority: China General Microbiological Culture Collection Center (CGMCC), Address: Institute of Microbiology, Chinese Academy of Sciences, No. 3, Courtyard 1, Beichen West Road, Chaoyang District, Beijing, China

Biological Material: SW-B106, Taxonomic Name: Parageobacillus caldoxylosilyticus, Deposit Number: CGMCC No. 31844, Deposit Date: Sep. 3, 2024, Depository Authority: China General Microbiological Culture Collection Center (CGMCC), Address: Institute of Microbiology, Chinese Academy of Sciences, No. 3, Courtyard 1, Beichen West Road, Chaoyang District, Beijing, China.
(4) Biological Material: SW-B107, Taxonomic Name: Aeribacillus pallidus, Deposit Number: CGMCC No. 31845, Deposit Date: Sep. 3, 2024, Depository Authority: China General Microbiological Culture Collection Center (CGMCC), Address: Institute of Microbiology, Chinese Academy of Sciences, No. 3, Courtyard 1, Beichen West Road, Chaoyang District, Beijing, China.
(5) Biological Material: SW-B109, Taxonomic Name: Pallidibacillus thermolactis, Deposit Number: CGMCC No. 31847, Deposit Date: Sep. 3, 2024, Depository Authority: China General Microbiological Culture Collection Center (CGMCC), Address: Institute of Microbiology, Chinese Academy of Sciences, No. 3, Courtyard 1, Beichen West Road, Chaoyang District, Beijing, China.

### BRIEF DESCRIPTION OF THE DRAWINGS

The present invention will be further described in detail below with reference to the attached drawings and examples.

FIG. 1 is a microscopic photograph of Bacillus smithii provided by the present invention.

FIG. 2 is a microscopic photograph of Heyndrickxia coagulans provided by the present invention.

FIG. 3 is a microscopic photograph of Parageobacillus caldoxylosilyticus provided by the present invention.

FIG. 4 is a microscopic photograph of Aeribacillus pallidus provided by the present invention.

FIG. 5 is a microscopic photograph of Pallidibacillus thermolactis provided by the present invention.

FIG. 6 is a schematic diagram of the activation and cultivation process provided by the present invention.

FIG. 7 is a schematic diagram after enrichment culture provided by the present invention.

FIG. 8 is one of the schematic diagrams of streaking isolation provided by the present invention.

FIG. 9 is the second schematic diagram of streaking isolation provided by the present invention.

FIG. 10 is a schematic diagram of the bacterial morphology observed by microscopic examination provided by the present invention.

FIG. 11 is a schematic diagram of the simulated small-scale pilot test process provided in the present invention.

FIG. 12 is a schematic diagram of the sludge concentration tracking in the biochemical system provided by Verification Example 1 of the present invention.

FIG. 13 is a schematic diagram of the excess sludge discharge tracking in the biochemical system provided by Verification Example 1 of the present invention.

FIG. 14 is a schematic diagram of the on-site photograph provided by Verification Example 1 of the present invention.

FIG. 15 is a schematic diagram of the sludge concentration tracking in the biochemical system provided by Verification Example 2 of the present invention.

FIG. 16 is a schematic diagram of the excess sludge discharge tracking in the biochemical system provided by Verification Example 2 of the present invention.

FIG. 17 is a schematic diagram of the on-site photograph provided by Verification Example 2 of the present invention.

### DETAILED DESCRIPTION

After long-term research, the inventors have successively screened, isolated, and purified five strains of high-efficiency sludge-reducing bacteria from the environment, which were numbered #HT1, #HT2, #HT3, #HT4, and #HT5 respectively. Through 16S rDNA sequencing, the sequencing results were compared with the NCBI GenBank database and the 16S rDNA bacterial database (Maidak et al, 2001). A phylogenetic tree was constructed using MEGA software to determine the genus and species of the bacteria, and analysis was performed to confirm the taxonomic classification of the strains.

A microscopic photograph of the morphological characteristics of the strain numbered #HT1 is shown in FIG. 1. 16S rDNA sequencing and comparison with the NCBI GenBank database indicated that this strain is Bacillus smithii. Subsequently, the strain was deposited in the China General Microbiological Culture Collection Center (CGMCC), with the deposit number: CGMCC No. 31841.

The >16S rDNA sequencing result of this strain is shown in SEQ ID No: 1: si

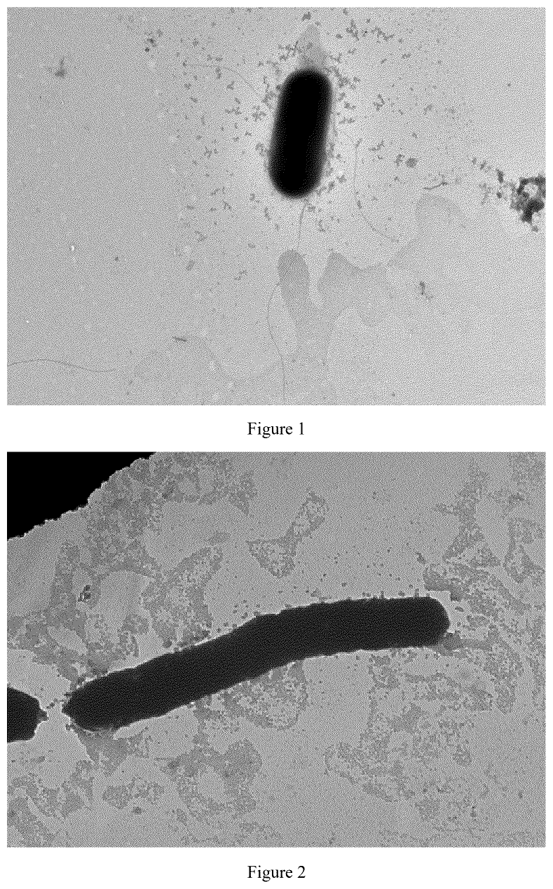

A microscopic photograph of the morphological characteristics of the strain numbered #HT2 is shown in FIG. 2. 16S rDNA sequencing and comparison with the NCBI GenBank database indicated that this strain is Heyndrickxia coagulans. Subsequently, the strain was deposited in the China General Microbiological Culture Collection Center (CGMCC), with the deposit number: CGMCC No. 31842.

The >16S rDNA sequencing result of this strain is shown in SEQ ID No: 2:
ttccagggcg cgtgctatac atgcaagtcg tgcggacctt ttaaaagctt gcttttaaaa 60
ggttagcggc ggacgggtga gtaacacgtg ggcaacctgc ctgtaagatc gggataacgc 120
cgggaaaccg gggctaatac cggatagttt tttcctccgc atggaggaaa aaggaaagac 180
ggcttctgct gtcacttaca gatgggcccg cggcgcatta gcttgttggt ggggtaacgg 240
ctcaccaagg caacgatgcg tagccgacct gagagggtga tcggccacat tgggactgag 300
acacggccca aactcctacg ggaggcagca gtagggaatc ttccgcaatg gacgaaagtc 360
tgacggagca acgccgcgtg agtgaagaag gccttcgggt cgtaaaactc tgttgccggg 420
gaagaacaag tgccgttcga acagggcggc gccttgacgg tacccggcca gaaagccacg 480
gctaactacg tgccagcagc cgcggtaata cgtaggtggc aagcgttgtc cggaattatt 540
gggcgtaaag cgcgcgcagg cggcttctta agtctgatgt gaaatcttgc ggctcaaccg 600
caagcggtca ttggaaactg ggaggcttga gtgcagaaga ggagagtgga attccacgtg 660
tagcggtgaa atgcgtagag atgtggagga acaccagtgg cgaaggcggc tctctggtct 720
gtaactgacg ctgaggcgcg aaagcgtggg gagcaaacag gattagatac cctggtagtc 780
cacgccgtaa acgatgagtg ctaagtgtta gagggtttcc gccctttagt gctgcagcta 840
acgcattaag cactccgcct ggggagtacg gccgcaaggc tgaaactcaa aggaattgac 900
gggggcccgc acaagcggtg gagcatgtgg tttaattcga agcaacgcga agaaccttac 960
caggtcttga catcctctga cctccctgga gacagggcct tccccttcgg gggacagagt 1020
gacaggtggt gcatggttgt cgtcagctcg tgtcgtgaga tgttgggtta agtcccgcaa 1080
cgagcgcaac ccttgacctt agttgccagc attcagttgg gcactctaag gtgactgccg 1140
gtgacaaacc ggaggaaggt ggggatgacg tcaaatcatc atgcccctta tgacctgggc 1200
tacacacgtg ctacaatgga tggtacaaag ggctgcgaga ccgcgaggtt aagccaatcc 1260
cagaaaacca ttcccagttc ggattgcagg ctgcaacccg cctgcatgaa gccggaatcg 1320
ctagtaatcg cggatcagca tgccgcggtg aatacgttcc cgggccttgt acacaccgcc 1380
cgtcacacca cgagagtttg taacacccga agtcggtgag gtaaccttta cggagccagc 1440
cgccgaaggt gaacagatat tcttaa 1466

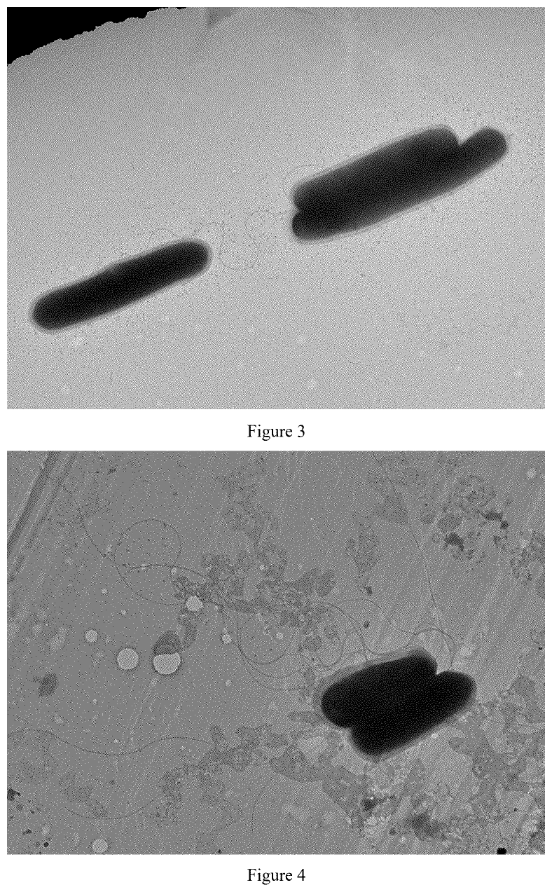

A microscopic photograph of the morphological characteristics of the strain numbered #HT3 is shown in FIG. 3. 16S rDNA sequencing and comparison with the NCBI GenBank database indicated that this strain is Parageobacillus caldoxylosilyticus. Subsequently, the strain was deposited in the China General Microbiological Culture Collection Center (CGMCC), with the deposit number: CGMCC No. 31844.

The >16S rDNA sequencing result of this strain is shown in SEQ ID No: 3:
catgatacgg cgtgctatac atgcagtcga gcggaccgaa caggagcttg ctcttgttcg 60
gttagcggcg gacgggtgag taacacgtgg gtaacctacc cgtaagaccg ggataactcc 120
gggaaaccgg agctaatacc ggataacacc aaagaccgca tggtctttgg ttgaaaggcg 180
gcttcggctg tcacttacgg atgggcccgc ggcgcattag ctagttggtg aggtaacggc 240
tcaccaaggc gacgatgcgt agccggcctg agagggtgac cggccacact gggactgaga 300
cacggcccag actcctacgg gaggcagcag tagggaatct tccgcaatgg acgaaagtct 360
gacggagcga cgccgcgtga gcgaagaagg tcttcggatc gtaaagctct gttgttaggg 420
aagaagaagt accgttcgaa tagggcggta cggtgacggt acctaacgag aaagccccgg 480
ctaactacgt gccagcagcc gcggtaatac gtagggggcg agcgttgtcc ggaattattg 540
ggcgtaaagc gcgcgcaggc ggtcccttaa gtctgatgtg aaagcccacg gctcaaccgt 600
ggagggtcat tggaaactgg gggacttgag tgcagaagag gagagcggaa ttccacgtgt 660
agcggtgaaa tgcgtagaga tgtggaggaa caccagtggc gaaggcggct ctctggtctg 720
taactgacgc tgaggcgcga aagcgtgggg agcaaacagg attagatacc ctggtagtcc 780
acgccgtaaa cgatgagtgc taagtgttag aggggtcaaa ccctttagtg ctgtagctaa 840
cgcgttaagc actccgcct 859

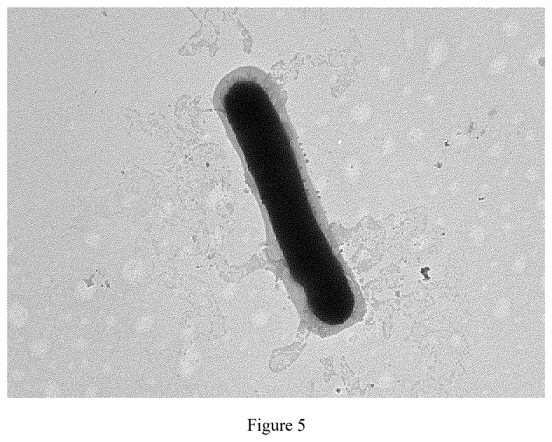

A microscopic photograph of the morphological characteristics of the strain numbered #HT4 is shown in FIG. 4. Comparison with the GenBank database indicated that this strain is Aeribacillus pallidus. Subsequently, the strain was deposited in the China General Microbiological Culture Collection Center (CGMCC), with the deposit number: CGMCC No. 31845.

The >16S rDNA sequencing result of this strain is shown in SEQ ID No: 4:
gttttagagg cgtgctatac atgcagtcga gcggaccgaa gggagcttgc tcctttaggt 60
tagcggcgga cgggtgagta acacgtgggc aacctgccct gcagactggg ataacttcgg 120
gaaaccggag ctaataccgg ataacaccga aaaccgcatg gttttcggtt gaaaggcggc 180
ttttagctgt cactgcagga tgggcccgcg gcgcattagc tagttggtga ggtaacggct 240
caccaaggcg acgatgcgta gccgacctga gagggtgacc ggccacactg ggactgagac 300
acggcccaga ctcctacggg aggcagcagt agggaatctt ccgcaatgga cgaaagtctg 360
acggagcaac gccgcgtgag cgaagaaggt cttcggatcg taaagctctg ttgtcaggga 420
agaacaagta ccgttcgaac agggcggtac cttgacggta cctgacgagg aagccacggc 480
taactacgtg ccagcagccg cggtaatacg taggtggcaa gcgttgtccg gaattattgg 540
gcgtaaagcg cgcgcaggcg gttccttaag tctgatgtga aatctcgcgg ctcaaccgcg 600
agcggccatt ggaaactggg gaacttgagt gcaggagagg ggagcggaat tccacgtgta 660
gcggtgaaat gcgtagagat gtggaggaac accagtggcg aaggcggctc tctggcctgt 720
aactgacgct gaggcgcgaa agcgtgggga gcgaacagga ttagataccc tggtagtcca 780
cgccgtaaac gatgagtgct aagtgttaga gggtatccac cctttagtgc tgcagcaaac 840
gcattaagca ctccgcctg 859

A microscopic photograph of the morphological characteristics of the strain numbered #HT5 is shown in FIG. 5. 16S rDNA sequencing and comparison with the NCBI GenBank database indicated that this strain is Pallidibacillus thermolactis. Subsequently, the strain was deposited in the China General Microbiological Culture Collection Center (CGMCC), with the deposit number: CGMCC No. 31847.

The >16S rDNA sequencing result of this strain is shown in SEQ ID No: 5:
gtgcagacgc gtgctataca tgcagtcgag cggaccaata gaaaagcttg cttttcttga 60
ggttagcggc ggacgggtga gtaacacgtg ggcaacctac ctgtaagact gggataactt 120
acggaaacgt gagctaatac cggatagttt cacttctcgc atgagaagtg aaggaaagat 180
ggcttttagc tatcacttac agatgggccc gcggcgcatt agctagttgg tggggtaaag 240
gcctaccaag gcgacgatgc gtagccgacc tgagagggtg atcggccaca ctgggactga 300
gacacggccc agactcctac gggaggcagc agtagggaat cttccgcaat ggacgaaagt 360
ctgacggagc aacgccgcgt gagcgaagaa ggtcttcgga tcgtaaagct ctgttgttag 420
ggaagaacaa gtaccggagt aactgacggt accttgacgg tacctaacca gaaagccacg 480
gctaactacg tgccagcagc cgcggtaata cgtaggtggc aagcgttgtc cggaatcatt 540
gggcgtaaag cgcgcgcagg cggtccttta agtctgatgt gaaatcttgc ggctcaaccg 600
taagcggtca ttggaaactg ggggacttga gtgcaggaga ggaaagcgga attccatgtg 660
tagcggtgaa atgcgtagag atatggagga acaccagtgg cgaaggcggc tttctggcct 720
gtaactgacg ctgaggcgcg aaagcgtggg gagcaaacag gattagatac cctggtagtc 780
cacgccgtaa acgatgagtg ctaagtgttg gagggtttcc gcccttcagt gctgcagcta 840
acgcattaag cactccgcc 859

Using the above five strains, with sludge reduction as the main evaluation index, five sets of compounding verification tests were conducted respectively. Finally, a microbial composite bacteria for sludge reduction in biochemical systems was invented. The microbial composite bacteria for sludge reduction in biochemical systems comprises the aforementioned Bacillus smithii, Heyndrickxia coagulans, Parageobacillus caldoxylosilyticus, Aeribacillus pallidus, and Pallidibacillus thermolactis. According to the ratio of effective viable bacteria count, the final ratio is: (10~25%):(25~30%):(18~26%):(12~29%):(9~30%).

Adding the aforementioned microbial composite bacteria for sludge reduction in biochemical systems into the biochemical system at a certain ratio can reduce the production of excess sludge in the biochemical system. The microbial composite bacteria for sludge reduction in biochemical systems decomposes various biological enzymes, and the synergistic metabolism among the strains decomposes the aged or dead sludge in the biochemical system into carbon dioxide and water. Meanwhile, through the competition between microorganisms, it inhibits the growth of some miscellaneous bacteria (such as filamentous bacteria-producing microorganisms), thereby achieving the purpose of sludge reduction.

Embodiments of the present invention provide a preparation method for microbial composite bacteria for sludge reduction in biochemical systems, comprising:

Step 1: Add sterile water to a sterile container containing soil material, thoroughly mix the soil material with the sterile water, and then allow the mixture to stand for sedimentation; wherein the soil material is surface moist soil or wet silt with apparent humus from a riverbank that has accumulated for over 20 years; For example, riparian wetland soils such as humic alluvial soils or organic gley soils.

Step 2: Take the supernatant, perform serial gradient dilution on the supernatant using the spread plate method to reduce the concentration of background miscellaneous bacteria; separately take the suspension of the supernatant at each dilution gradient and evenly spread it on NB solid medium, LB solid medium, NA solid medium, TSA solid medium, and PDA solid medium, then incubate them at a constant temperature of 40° C. in an incubator; take the undiluted supernatant and evenly spread it on NB solid medium, LB solid medium, NA solid medium, TSA solid medium, and PDA solid medium as a control, then incubate it at a constant temperature of 37° C. in an incubator.

Step 3: After single colonies grow, observe the bacterial isolation on all dilution gradient plates, and visually screen out a target gradient whose isolation effect meets the task requirements; select colonies with different apparent morphologies from the target gradient and pick single colonies. Perform serial gradient dilution on the single colonies using the spread plate method, then evenly spread them on the corresponding medium and incubate at a constant temperature of 35° C. in an incubator; evenly spread the undiluted single colonies on the corresponding medium as a control and incubate at a constant temperature of 35° C. in the incubator. Increase the incubator temperature by 1° C. incrementally and repeat this step until the incubator temperature reaches 45° C.; repeat this step again until the colony morphology of each single colony is consistent. Among them, the apparent morphology preferably includes the color, size, and shape of the colonies; in this step, the culture is carried out within the temperature range of 35° C.-45° C. where microorganisms have the strongest activity, and the method of incremental temperature culture is intended to distinguish the growth rates of different single colonies.

Step 4: Purify the endophytic bacteria using the streak plate method to obtain monoclonal colonies. After 16S rDNA gene sequencing, the microbial composite bacteria for sludge reduction in biochemical systems are obtained.

In Step 3, the formula of the corresponding medium includes:

Formulation of the Culture Medium for Bacillus smithii:
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;
   Formulation of the Culture Medium for Heyndrickxia coagulans:
NB Solid Medium: Peptone 20 g/L, Beef Extract 3 g/L, Agar Powder 20 g/L;
NB Liquid Medium: Peptone 20 g/L, Beef Extract 3 g/L;
After preparation, sterilize by autoclaving at 121° C. for 20 minutes;
Formulation of the culture medium for Parageobacillus caldoxylosilyticus;
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;
   Formulation of the Culture Medium for Aeribacillus pallidus:
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;
   Formulation of the Culture Medium for Pallidibacillus thermolactis:
LB Solid Medium: Peptone 10 g/L, Yeast Extract 5 g/L, Sodium Chloride 5 g/L, and Agar Powder 20 g/L;
LB Liquid Medium: Peptone 10 g/L, Yeast Extract 5 g/L, and Sodium Chloride 5 g/L;
After preparation, sterilize by autoclaving at 121° C. for 20 minutes;
   The Specific Experimental Process in this Example is as Follows:

### I. Preparation of Materials:

1. Prepare the required quantity of sterile 10 mL centrifuge tubes as needed;
2. Sterilize one bottle of sterile water and aliquot it into 10 mL sterile centrifuge tubes; preferably: sterilize one 500 mL bottle of sterile water, and add 5 mL of sterile water to each centrifuge tube.
3. Prepare sterilized spreaders or yellow sterile pipette tips for dilution, as well as 3 sterilized 100 mL Erlenmeyer flasks.
4. Prepare a constant-temperature incubator and a constant-temperature shaker. The temperature range of the shaker and the constant-temperature incubator shall be capable of reaching 45° C.

### II. The Preparation Method Comprises:

Soak the soil sample in sterile water using a sterile Erlenmeyer flask. After thoroughly mixing the soil sample with the sterile water, let the mixture stand for sedimentation. Collect the supernatant and perform serial 10-fold dilutions to obtain concentrations of 10⁻¹, 10⁻², and 10⁻³. Separately take 200 μL of the undiluted supernatant or the suspensions of the three dilution gradients, and evenly spread them onto five types of solid media: NB (Nutrient Broth) solid medium, LB (Luria-Bertani) solid medium, NA (Nutrient Agar) solid medium, TSA (Tryptic Soy Agar) solid medium, and PDA (Potato Dextrose Agar) solid medium.

Incubate at a constant temperature of 40° C. in an incubator, and place the control group in an incubator at 37° C. After 2-3 days, when monoclonal colonies grow, observe the bacterial isolation on the plates of the four dilution gradients. Visually select one gradient with good isolation effect for the subsequent endophytic bacteria isolation of the samples.

Based on the colony color, size, and shape, select strains with different apparent morphologies, pick the monoclonal colonies for cultivation, and number them.

Dilute and spread onto the corresponding medium. Place the corresponding medium in an incubator at 35° C.-45° C. for cultivation (with the control group also incubated in an incubator at 35° C.-45° C.) until the colony morphology is consistent. Purify the endophytic bacteria using the streak plate method to obtain monoclonal colonies, which are then subjected to 16S rDNA gene sequencing.

The invention discloses a microbial agent for sludge reduction in biochemical systems, comprising Bacillus smithii, Heyndrickxia coagulans, Parageobacillus caldoxylosilyticus, Aeribacillus pallidus, Pallidibacillus thermolactis, and a microbial composite bacteria for sludge reduction in biochemical systems (or the microbial composite bacteria for sludge reduction in biochemical systems obtained by a preparation method).

The present invention also provides the application of Bacillus smithii, Heyndrickxia coagulans, Parageobacillus caldoxylosilyticus, Aeribacillus pallidus, Pallidibacillus thermolactis, a microbial composite bacteria for sludge reduction in biochemical systems, the microbial composite bacteria for sludge reduction in biochemical systems obtained by the preparation method, or a microbial agent for sludge reduction in biochemical systems in sludge reduction of biochemical systems.

The present invention discloses a microbial composite bacterium for biochemical system sludge reduction, a method for preparing the same, the microbial composite bacterium for biochemical system sludge reduction obtained by the preparation method, or a method for using a microbial agent for biochemical system sludge reduction in a biochemical system, comprising:

Take the composite microbial bacteria for sludge reduction in biochemical systems or the microbial agent for sludge reduction in biochemical systems and add it to the biochemical system to mix with the sludge.

Among them, the Dosing Methods Include:

The temperature of the biochemical system shall be within the range of 25° C. to 45° C., which is adaptable to the actual ambient temperature range of the biochemical system throughout the four seasons. Preferably, it is between 35° C. and 45° C., as the microbial activity of the microbial composite bacteria for sludge reduction in biochemical systems or the microbial agent for sludge reduction in biochemical systems is the strongest under this temperature condition; the DO (Dissolved Oxygen) value shall be between 1.0 mg/L and 4.0 mg/L, and within this DO value range, anaerobic bacteria are predominant in both the surface layer and the inner layer; the pH value shall be between 6 and 9. Beyond this range, the microbial composite bacteria for sludge reduction in biochemical systems or the microbial agent for sludge reduction in biochemical systems will become inactive.
The dosing points are: the inlet end of the aerobic tank in the biochemical system and the sludge return point of the secondary clarifier;
To reduce 1.0 ton of absolute dry sludge in the biochemical system, the dosage of the composite microbial bacteria for sludge reduction in biochemical systems or the microbial agent for sludge reduction in biochemical systems is 2.5~5 kg;
Determine the daily dosage of the microbial composite bacteria for sludge reduction in biochemical systems or the microbial agent for sludge reduction in biochemical systems based on the excess sludge production derived from the daily influent load and output of the biochemical system.

Among them, the temperature at which the microbial composite bacteria for sludge reduction in biochemical systems or the microbial agent for sludge reduction in biochemical systems exhibit the strongest microbial activity ranges from 35° C. to 45° C., and the preferred temperature of the biochemical system is 35° C. to 45° C.

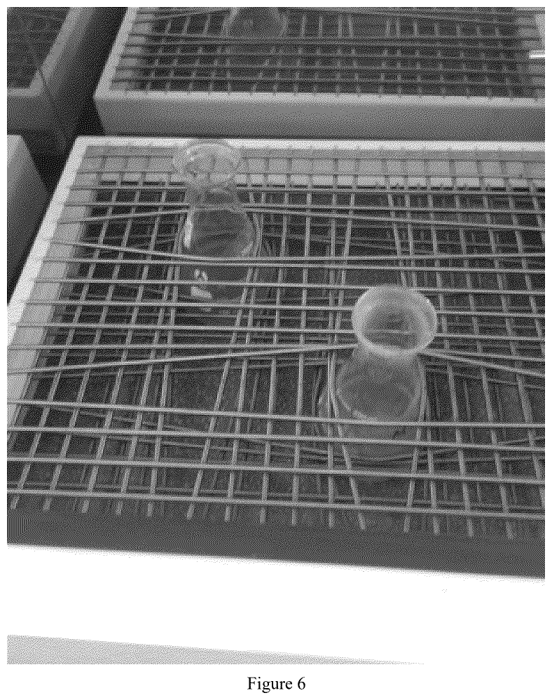

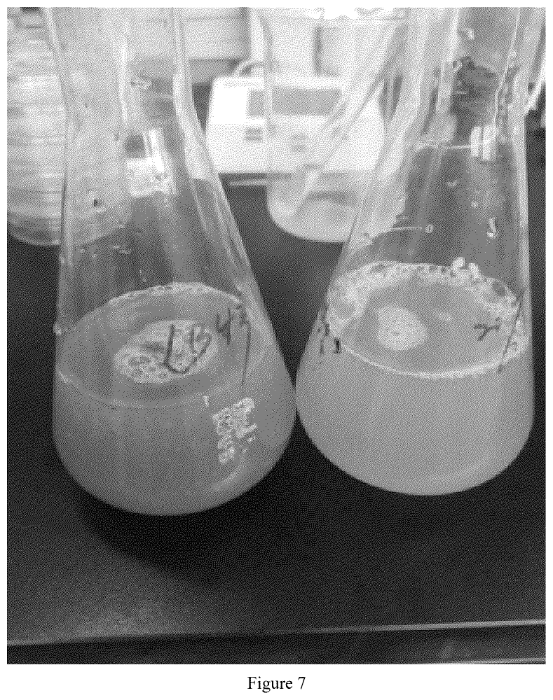

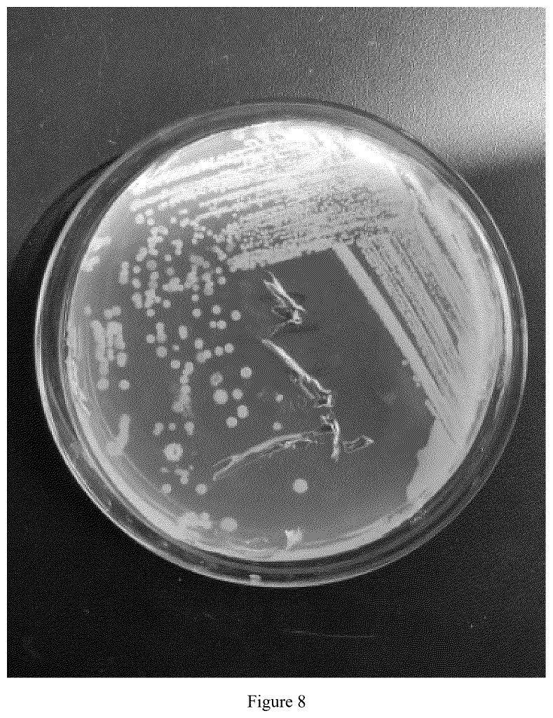

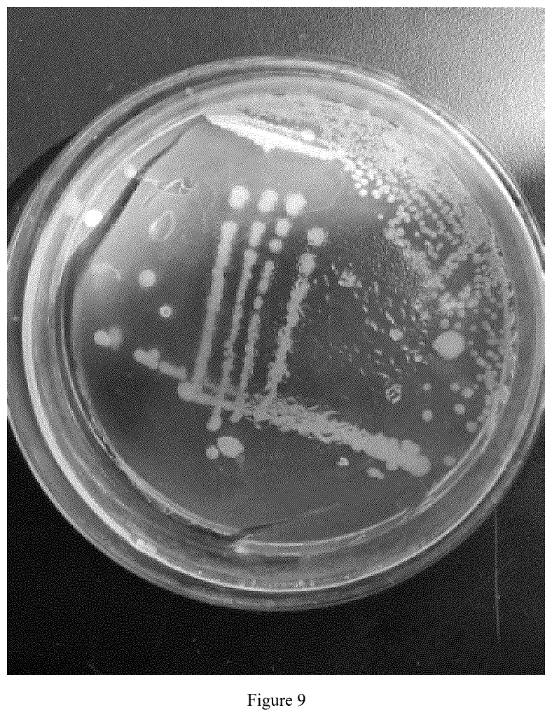

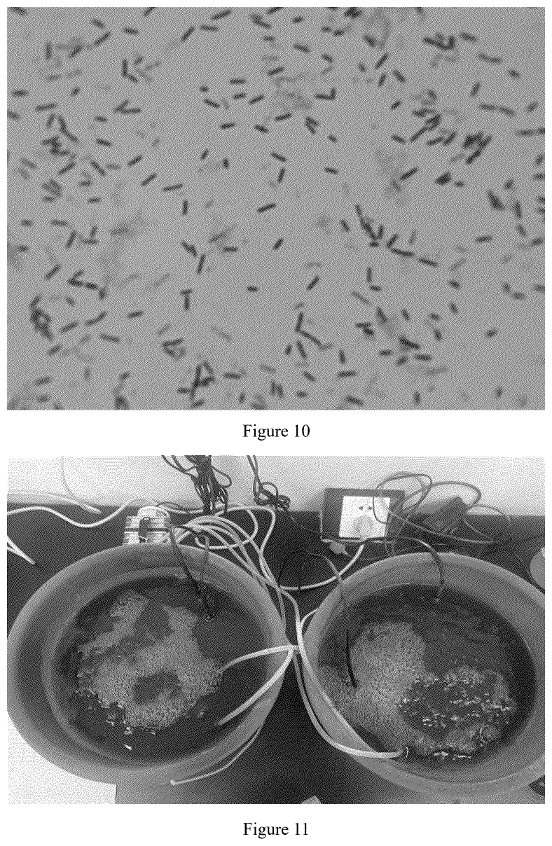

As shown in FIGS. 6 to 11, to conduct on-site verification of the technical solution of the present invention, two specific verification examples are provided below:

First, carry out the preparation work as follows:
I. Analysis of Excess Sludge Yield
II. Preparation of the microbial composite bacteria for sludge reduction in biochemical systems or the microbial agent for sludge reduction in biochemical systems
III. Determine the optimal dosage based on the sludge properties.
IV. Optimal Reduction Environment and Effect Tracking
V. The project implementation achieves sludge reduction.

### Verification Example 1: A Municipal Sewage Treatment Enterprise

1.1 Preliminary Preparation: Based on the analysis of the sludge properties in the biochemical system, its MLSS concentration is 11,000 mg/L, MLVSS is 7,150 mg/L, and the VSS/TSS ratio is 0.625; the daily sludge discharge of the biochemical system is approximately 15 tons with a moisture content of around 85%.

### 1.2 Preparation of Composite Bacterial Strains

Preparation of Experimental Bacterial Suspension: Scrape 1 inoculating loop of bacteria from the slant culture, inoculate it into the sterilized LB medium, and incubate with shaking for 24 hours.

Experimental Instruments and Materials: Shaker, bench-scale aeration device, dissolved oxygen detector, experimental containers, index detection instruments, activated sludge, and sewage from the enterprise for testing (used to evaluate the sludge reduction effect of different bacterial strains).

### 1.3 Experimental Procedures

Place 1000 mL of aerobic activated sludge in a bench-scale experimental device, add 2000 mL of sludge-water mixture and 10 mL of the cultured bacterial suspension, and install the aeration device;

Start the aeration device and maintain the dissolved oxygen concentration at approximately 2-5 mg/L;

Conduct continuous aeration for the reaction, collect the sludge-water mixture at intervals during the process, and determine the decrease in the MLSS index.

Conduct experiments with different bacterial strains under the same conditions and at the same time.

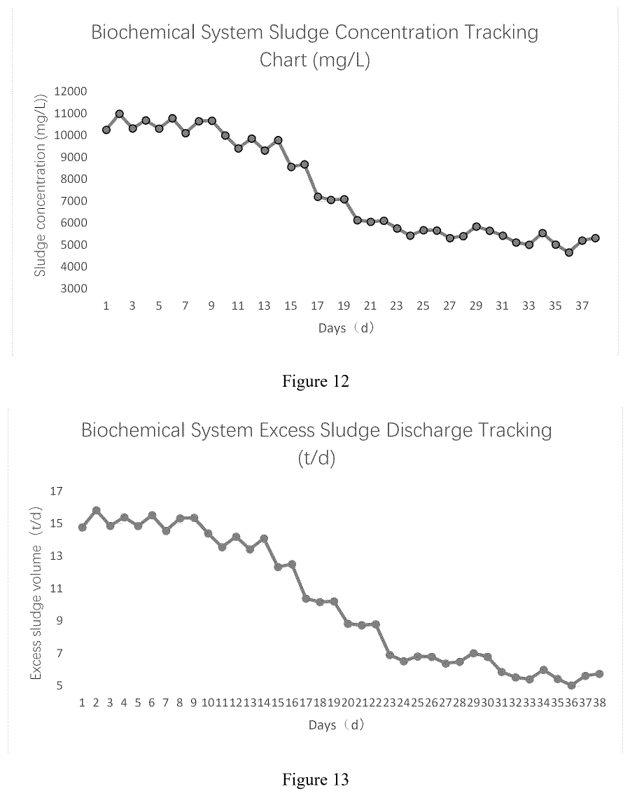

According to the test results, 80 kg of the composite bacteria is added daily, and the on-site tracking and analysis of its data changes are shown in FIGS. 12 to 13.

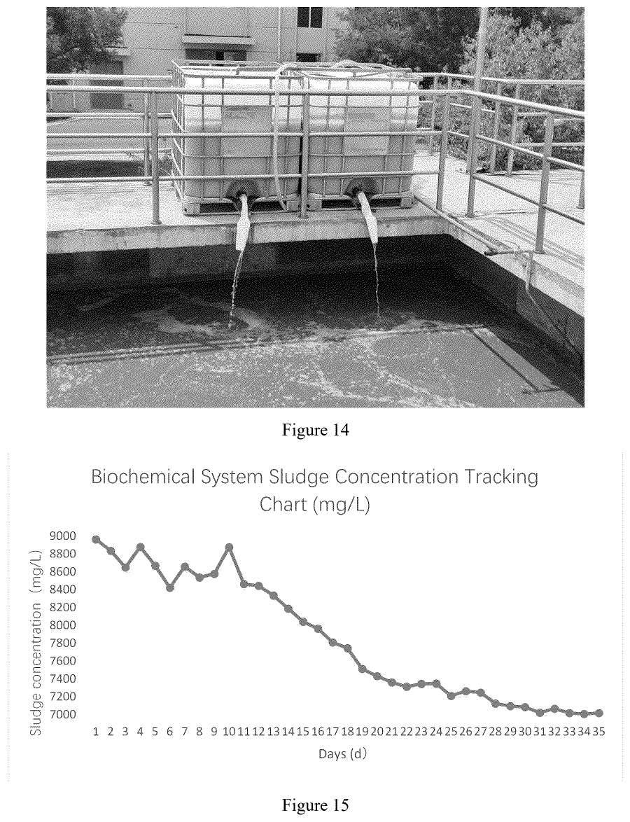

As shown in FIG. 14, the sewage treatment plant of this municipal sewage enterprise has a daily water volume of approximately 50,000 tons and adopts the A²/O biological treatment process. The sludge yield of its biological system is about 21 tons (with a moisture content of 85%) per 10,000 tons of water; the test results are shown in Table 1.

TABLE 1

| | | MLSS | |
|---|---|---|---|
| Test | Proportion | Concentration | |
| Groups | of Strains | Data | Efficacy |
| 1 | 20%:20%:20%:20%:20% | 9500 | +++ |
| 2 | 20%:25%:20%:15%:20% | 8050 | +++++ |
| 3 | 25%:25%:25%:10%:15% | 8250 | ++++ |
| 4 | 10%:30%:10%:25%:25% | 9000 | + |
| 5 | 20%:20%:25%:20%:15% | 8350 | +++ |

Note:
The order of the bacterial strains is as follows: Bacillus smithii, Heyndrickxia coagulans, Parageobacillus caldoxylosilyticus, Aeribacillus pallidus, and Pallidibacillus thermolactis.

### Experimental Results and Analysis

In the sludge reduction test with the addition of Bacillus smithii, Heyndrickxia coagulans, Parageobacillus caldoxylosilyticus, Aeribacillus pallidus, and Pallidibacillus thermolactis, after 24 hours of aeration during the reaction, the optimal strain ratio was determined as (20%):(25%):(20%):(15%):(20%) through the analysis of MLSS and MLVSS.

### Verification Example 2: A Case of Sludge Reduction in Industrial Wastewater

2.1 On-site Situation Collection: Preliminary Preparation: Based on the analysis of the system's sludge properties, its MLSS concentration is 9,000 mg/L, MLVSS is 5,400 mg/L, and the VSS/TSS ratio is 0.6; the daily sludge discharge of the biochemical system is approximately 18 tons with a moisture content of around 80%.

### 2.2 Experimental Preparation

Preparation of Experimental Bacterial Suspension: Scrape one inoculating loop of bacteria from the slant culture, inoculate it into the sterilized LB medium, and incubate with shaking for 24 hours.

Experimental Instruments and Materials: Shaker, bench-scale aeration device, dissolved oxygen detector, experimental containers, index detection instruments, activated sludge, and industrial wastewater from this project for experimental use.

### 2.3 Experimental Procedures

Place 1000 mL of aerobic activated sludge in a bench-scale experimental device, add 2000 mL of sludge-water mixture and 10 mL of the cultured bacterial suspension, and install the aeration device;
Start the aeration device and maintain the dissolved oxygen concentration at approximately 2-5 mg/L;
Conduct a continuous aeration reaction, collect the sludge-water mixture at intervals during the process, and determine the MLSS index;
Conduct experiments with different bacterial strains under the same conditions and at the same time.

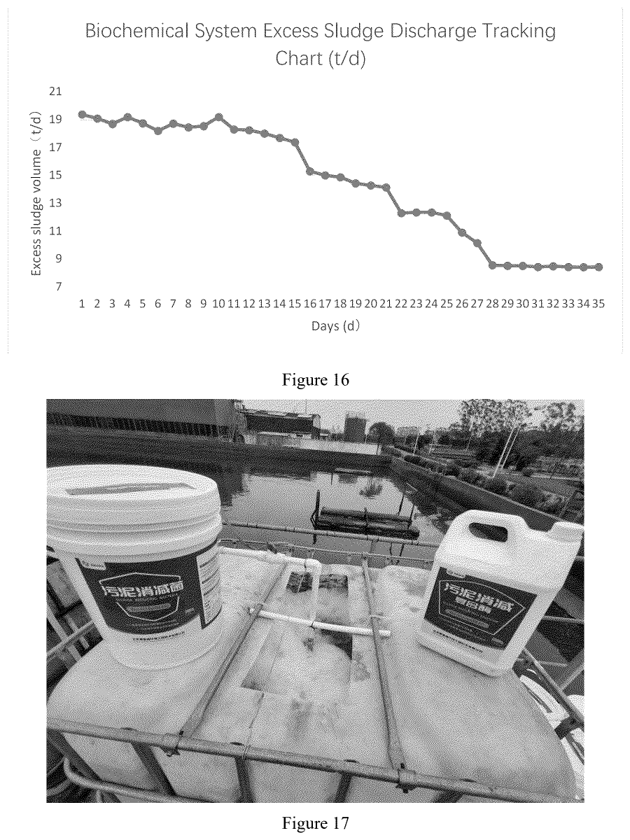

According to the test results, 5 kg of the composite bacteria is added daily, and the on-site tracking and analysis of its data changes are shown in FIGS. 15 to 16.

As shown in FIG. 17, this industrial sewage treatment plant has a daily water volume of approximately 20,000 tons/day and adopts the SBR (Sequencing Batch Reactor) treatment process in its biochemical system. The daily production of residual sludge (dry solids) is about 1.0 ton, equivalent to 5 tons/day of sludge with a moisture content of 80%. The comprehensive disposal cost of sludge with 80% moisture content is approximately 800 yuan/ton, resulting in a daily disposal cost of 4,000 yuan. The dosage of the composite bacterial strains is 5 kg per day, and the test results are shown in Table 2.

TABLE 2

| | | MLSS | |
|---|---|---|---|
| Test | Proportion | Concentration | |
| Groups | of Strains | Data | Efficacy |
| 1 | 20%:20%:20%:20%:20% | 7900 | +++ |
| 2 | 20%:25%:25%:10%:20% | 8100 | + |
| 3 | 20%:25%:25%:15%:15% | 7250 | ++++ |
| 4 | 10%:30%:10%:25%:25% | 8000 | ++ |
| 5 | 20%:20%:25%:20%:15% | 7150 | +++++ |

Note: The order of the bacterial strains is as follows: Bacillus smithii, Heyndrickxia coagulans, Parageobacillus caldoxylosilyticus, Aeribacillus pallidus, and Pallidibacillus thermolactis.

### Experimental Results and Analysis

In the sludge reduction test with the addition of Bacillus smithii, Heyndrickxia coagulans, Parageobacillus caldoxylosilyticus, Aeribacillus pallidus, and Pallidibacillus thermolactis, after 24 hours of aeration during the reaction, the optimal strain ratio was determined as (20%):(20%):(25%):(20%):(15%) through the analysis of MLSS and MLVSS.

In summary, the beneficial effects of the embodiments of the present invention are as follows:

Through extensive experimental verification, the ability of the microbial strains in the microbial composite bacteria for sludge reduction in biochemical systems or the microbial agent for sludge reduction in biochemical systems disclosed in the present invention to secrete different enzymes under specific environments is as follows:
Bacillus smithii secretes protease and amylase;
Heyndrickxia coagulans secretes L-lactate dehydrogenase, amylase, and protease;
Parageobacillus caldoxylosilyticus secretes xylanase and cellulase;
Aeribacillus pallidus secretes 4-α-glucosyltransferase, amylase, and protease;
Pallidibacillus thermolactis secretes glycolytic enzymes and lactate dehydrogenase.

The aforementioned microbial strains and different enzymes act to decompose various component substances of aged or dead microbial cells in the biochemical system, thereby providing energy for the microbial composite bacteria/microbial agent. The decomposed organic matrix of the sludge microbial cells produces carbon dioxide and water, or partially adsorbed organic matrices in the sludge are decomposed and converted into carbon dioxide and water, thus achieving sludge reduction. Meanwhile, through microbial competition, the growth of certain undesirable bacteria, such as filament-producing microorganisms, is inhibited.

By adding the microbial composite bacteria for sludge reduction in biochemical systems or the microbial agent for sludge reduction in biochemical systems disclosed in the present invention into the biochemical system, the present invention reduces the produced excess sludge, thereby achieving sludge reduction control at the source. The technical solution disclosed in the present invention has the following advantages:
1. No hardware investment is required, featuring extremely low energy consumption and relatively low investment and operation costs;
2. It can reduce the excess sludge in the biochemical system by 30% to 80%;
3. For systems requiring additional carbon sources, the supplementary amount of carbon sources can be reduced;
4. The sludge after reduction is more amenable to dewatering or disposal.

The technical solution disclosed in the present invention is applicable to sewage treatment plants handling substances with high organic content and no obvious toxicity. It is also suitable for the centralized disposal of multiple sewage plants and the co-treatment of various organic wastes, featuring high activity, low investment, low energy consumption, convenient operation, and no secondary pollution.

Compared with the prior art, the advantages of the present invention over the prior art are shown in Table 3:

TABLE 3

| This invention patent | prior art |
|---|---|
| The sludge reduction rate ranges | It only reduces the moisture |
| from 30% to 80%. | content of sludge. |
| No hardware investment required | entails substantial investment |
| | in hardware |
| Operating cost: 500 yuan per | The cost per ton of dry solids |
| ton of dry solids sludge | sludge exceeds 1,500 yuan. |
| Extremely low energy consumption | High energy consumption |

As mentioned above, the foregoing are merely specific embodiments of the present invention, but the protection scope of the present invention is not limited thereto. Any person skilled in the art within the technical field disclosed by the present invention can easily conceive of changes or substitutions, which shall all be included within the protection scope of the present invention. Therefore, the protection scope of the present invention shall be subject to the protection scope defined by the appended claims.

1. A microbial composite consortium comprising:
Bacillus smithii,
Heyndrickxia coagulans,
Parageobacillus caldoxylosilyticus,
Aeribacillus pallidus, and
Pallidibacillus thermolactis.

2. The microbial composite consortium of claim, wherein an effective viable count ratio of Bacillus smithii:Heyndrickxia coagulans:Parageobacillus caldoxylosilyticus Aeribacillus pallidus:Pallidibacillus thermolactis is (10-25%):(25-30%):(18-26%):(12-29%):(9-30%).

3. The microbial composite consortium of claim, wherein the Bacillus smithii is deposited at the China General Microbiological Culture Collection Center (CGMCC), with a deposition number CGMCC No. 31841.

4. The microbial composite consortium of claim, wherein the Heyndrickxia coagulans is deposited at the China General Microbiological Culture Collection Center (CGMCC), with a deposition number CGMCC No. 31842.

5. The microbial composite consortium of claim, wherein the Parageobacillus caldoxylosilyticus is deposited at the China General Microbiological Culture Collection Center (CGMCC), with a deposition number CGMCC No. 31844.

6. The microbial composite consortium of claim, wherein the Aeribacillus pallidus is deposited at the China General Microbiological Culture Collection Center (CGMCC), with a deposition number CGMCC No. 31845.

7. The microbial composite consortium of claim, wherein the Pallidibacillus thermolactis is deposited at the China General Microbiological Culture Collection Center (CGMCC), with a deposition number CGMCC No. 31847.

8. A method for preparing the microbial composite consortium of claim, the method includes:
adding sterile water to a sterile container containing soil material, wherein the soil material is surface moist soil or wet silt with apparent humus from a riverbank that has accumulated for over 20 years;
thoroughly mixing the soil material with the sterile water;
letting the mixture stand for sedimentation to generate a sedimented mixture;
taking some of an undiluted supernatant of the sedimented mixture, and evenly spreading the undiluted supernatant (serving as the control group) on Nutrient Broth Agar (NB) solid medium, Luria-Bertani Agar (LB) solid medium, Nutrient Agar (NA) solid medium, Tryptic Soy Agar (TSA) solid medium, and Potato Dextrose Agar (PDA) solid medium, and placing these media in an incubator for constant-temperature cultivation at 37° C.;
taking the remaining supernatant of the sedimented mixture and performing serial dilution on the supernatant using the dilution plating method, then
separately taking a suspension of the supernatant at each dilution gradient and evenly spreading it on NB solid medium, LB solid medium, NA solid medium, TSA solid medium, and PDA solid medium, and placing these media in an incubator for constant-temperature cultivation at 40° C.; and
after formation of single colonies, observing the bacterial separation across all dilution gradient plates and visually identifying and selecting one target gradient where the separation effectiveness meets the requirements;
selecting colonies with different morphological appearances from the target gradient, picking the single colonies, performing serial dilution on the single colonies using the dilution plating method, then evenly spreading them on the corresponding medium, and placing the medium in an incubator for constant-temperature cultivation at 35° C.; and
for the control group of the undiluted supernatant, evenly spreading the undiluted single colonies on the corresponding medium, placing them in the incubator for constant-temperature cultivation at 35° C., and then increasing the incubator temperature by 1° C. incrementally;
repeating this step until the incubator temperature reaches 45° C.; repeating this entire process again until the morphological characteristics of each single colony become consistent;
using the streaking method for isolation to purify the endophytic bacteria and obtain monoclonal colonies; after 16S rDNA gene sequencing, the microbial composite bacterium for sludge reduction in biochemical systems is obtained.

9. The method of claim, wherein the method further includes an effective viable count ratio of Bacillus smithii:Heyndrickxia coagulans:Parageobacillus caldoxylosilyticus:Aeribacillus pallidus:Pallidibacillus thermolactis is (10-25%):(25-30%):(18-26%):(12-29%):(9-30%).

10. The method of claim, wherein the formulations of the corresponding culture medium for Bacillus smithii are:
LB Solid Medium: Peptone 10 g/L, Yeast Extract 5 g/L, Sodium Chloride 5 g/L, and Agar Powder 20 g/L; and
LB Liquid Medium: Peptone 10 g/L, Yeast Extract 5 g/L, and Sodium Chloride 5 g/L; and
wherein after preparation, the media are sterilized by autoclaving at 121° C. for 20 minutes.

11. The method of claim, wherein the formulations of the corresponding culture medium for Heyndrickxia coagulans are:
NB Solid Medium: Peptone 20 g/L, Beef Extract 3 g/L, Agar Powder 20 g/L; and
NB Liquid Medium: Peptone 20 g/L, Beef Extract 3 g/L; and
wherein after preparation, the media are sterilized by autoclaving at 121° C. for 20 minutes.

12. The method of claim, wherein the formulations of the corresponding culture medium for Parageobacillus caldoxylosilyticus are:
LB Solid Medium: Peptone 10 g/L, Yeast Extract 5 g/L, Sodium Chloride 5 g/L, and Agar Powder 20 g/L; and
LB Liquid Medium: Peptone 10 g/L, Yeast Extract 5 g/L, and Sodium Chloride 5 g/L; and
wherein after preparation, the media are sterilized by autoclaving at 121° C. for 20 minutes.

13. The method of claim, wherein the formulations of the corresponding culture medium for Aeribacillus pallidus are:
LB Solid Medium: Peptone 10 g/L, Yeast Extract 5 g/L, Sodium Chloride 5 g/L, and Agar Powder 20 g/L; and
LB Liquid Medium: Peptone 10 g/L, Yeast Extract 5 g/L, and Sodium Chloride 5 g/L; and
wherein after preparation, the media are sterilized by autoclaving at 121° C. for 20 minutes.

14. The method of claim, wherein the formulations of the corresponding culture medium for Pallidibacillus thermolactis are:
LB Solid Medium: Peptone 10 g/L, Yeast Extract 5 g/L, Sodium Chloride 5 g/L, and Agar Powder 20 g/L; and
LB Liquid Medium: Peptone 10 g/L, Yeast Extract 5 g/L, and Sodium Chloride 5 g/L; and
wherein after preparation, the media are sterilized by autoclaving at 121° C. for 20 minutes.

15. A method for preparing a microbial composite agent from the microbial composite consortium of claim 1 for sludge reduction in a biochemical system, wherein the method includes:
taking the microbial composite consortium for sludge reduction in a biochemical system; and adding the microbial composite consortium to the biochemical system to mix with the sludge by determining a daily dosage of the microbial composite consortium for sludge reduction in the biochemical system based on an excess sludge production derived from both the daily influent load and output of the biochemical system.

16. The method of claim, wherein the temperature of the biochemical system is maintained between 25° C. to 45° C.

17. The method of claim, wherein a Dissolved Oxygen (DO) value is between 1.0 mg/L and 4.0 mg/L.

18. The method of claim, wherein a pH value is between 6 to 9.

19. The method of claim 15, wherein dosing points are located at an inlet end of an aerobic tank in the biochemical system and a sludge return point of a secondary clarifier.

20. The method of claim, wherein dosage of the composite microbial bacteria for sludge reduction in the biochemical system is 2.5~5 kg, capable of reducing 1.0 ton of absolute dry sludge in the biochemical system.
